(19) United States
(12) Patent Application Publication
(10) Pub. No.: US 2004/0122493 A1
(43) Pub. Date: Jun. 24, 2004

(54) Ultrasonic irradiation apparatus

(75) Inventors: Yoshiharu Ishibashi, Tochigi-Ken (JP); Katsuhiko Fujimoto, Saitama-ken (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: KABUSHIKI KAISHA TOSHIBA, Tokyo (JP)

(21) Appl. No.: 10/657,084

(22) Filed: Sep. 9, 2003

(30) Foreign Application Priority Data
Sep. 9, 2002 (JP) P2002-263062
Sep. 26, 2002 (JP) P2002-280590
Oct. 29, 2002 (JP) P2002-313673

(51) Int. Cl. A61F 7/00; A61F 7/12

(52) U.S. Cl. 607/96; 600/439

(57) ABSTRACT

An ultrasound irradiation apparatus which irradiates an ultrasonic wave to a desired area by selecting two or more piezoelectric elements and by changing the selected piezoelectric elements. The selection may be performed by mechanical movement of substrates or electrical switches. A size of an aperture defined by selected irradiating piezoelectric elements may be determined based on a focal distance of an ultrasonic wave focusing is performed by varying delay times of drive signals applied to the selected piezoelectric elements.

### CROSS-REFERENCE TO RELATED APPLICATIONS

This application is based upon and claims the benefit of priority of prior Japanese Patent Applications No. 2002-280590 filed on Sep. 26, 2002, No. 2002-313673 filed on Oct. 29, 2002, and No. 2002-263062 filed on Sep. 9, 2002, the entire contents of which are incorporated herein by reference.

### FIELD OF THE INVENTION

The present invention relates to an ultrasonic irradiation apparatus which irradiates an ultrasonic wave toward an object.

### BACKGROUND OF THE INVENTION

In recent years, low invasive medical treatment attracts attention and has been tried to treat a cancer. Since the cancer is conventionally treated by a surgical operation, such as by surgical removal of tissue including the cancer, an original function of an internal organ can decrease, or appearance can be disfigured. In such cases, much burden is imposed on a patient, even if a life is supported. When QOL (quality of life) is considered, development of a low invasive medical treatment apparatus is desired strongly, in lieu of the conventional surgical operation. One low invasive medical treatment under development includes a method for heating the tissue by irradiating a strong ultrasonic wave toward the tissue to necrotize the cancer. In such method, although it is desired to heat a whole cancer whose diameter is 5 mm to 10 mm, for example, with uniform energy, the energy of the strong ultrasonic wave is concentrated on an area whose diameter is 2 mm to 3 mm, by a conventional method for irradiating the ultrasonic wave. Thus, it is difficult to heat the whole cancer by the strong ultrasonic wave. In order to solve this problem, a phased array technique which controls a phase of a driving signal for generating the strong ultrasonic wave from 2000 to 3000 piezoelectric elements to electrically set a position of focus of the ultrasonic wave, is being developed. However, it is difficult to realize the phased array technique, since the apparatus can be complicated.

For this reason, a method for irradiating and distributing the ultrasonic wave to the cancer with fewer piezoelectric elements and a simple driving circuit, has been proposed in Japanese Patent Publication (Kokai) No. 2000-166940. In this method, a generating unit of the strong ultrasonic wave has 4 to 24 piezoelectric elements. A first driving signal is supplied to a first piezoelectric elements group selected from these piezoelectric elements, and a second driving signal whose phase is different from the first driving signal by an arbitrary angle of 180 or less degrees, is supplied to a second piezoelectric elements group formed by the remaining piezoelectric elements. Thus, two or more maximum points of sound pressure are formed in the cancer, and ultrasonic energy is distributed. According to this method, it is possible to extend an irradiation range of the ultrasonic wave with fewer piezoelectric elements and the simple driving circuit.

Another method for irradiating the strong ultrasonic wave and for mechanically moving a concave generating unit of the strong ultrasonic wave, has been proposedin Japanese Patent Publication (Kokai) No. 11-226046. In this method, since the focus position of the strong ultrasonic wave moves arbitrarily on an orbit and the range or distance of the movement is arbitrarily set, it is possible to uniformly heat the cancer irrespective of its size and form.

However, according to the first method as disclosed in the Japanese Patent Publication (Kokai) No. 2000-166940, there are some problems. For instance, since the piezoelectric elements are few, an arrangement distance between the piezoelectric elements is large, or since the phase is limited to two angles, such as 0° and an arbitrary angle 180° or less degrees, accuracy of phase arrangement is low. For these reasons, since it is difficult to arrange a wave face of the ultrasonic wave, non-permissible heat areas (sub-maximum points) can be created. It is also difficult to produce a uniform sound pressure distribution in the irradiation range.

On the other hand, according to the method disclosed in Japanese Patent Publication (Kokai) No. 11-226046, when the generating unit of the strong ultrasonic wave mechanically moves to extend the irradiation range, coupling film attached to the generating unit, coupling liquid contained in the coupling film and also an ultrasonic imaging probe move. For this reason, the imaging mechanism can be complicated or enlarged. Moreover, by this method, in treating a liver cancer, an incidence space of the ultrasonic wave to the patient can be closed by a costa near a body surface when the generating unit moves. For this reason, a part of the strong ultrasonic wave cannot reach the predetermined area to be heated, and a heat area can be created near the costa. In addition, since an ultrasonic wave transmitted or received by an ultrasonic imaging apparatus is influenced by the costa, sound shade or multiplex reflection is created on the ultrasonic image, and quality of the image decreases.

For example, as shown in FIG. 1A, after irradiating a strong ultrasonic wave such that a focus point 9 is created in a right part of a cancer 2 located behind a costa 3 of a patient 1, a strong ultrasonic wave generating unit 6 moves along a surface of the patient 1 (horizontal direction) by ΔX such that a left part of the cancer 2 is irradiated, as shown in FIG. 11B. In such a case, since an ultrasonic imaging probe 5 is located in a center of the strong ultrasonic wave generating unit 6, the costa 3 is included in a ultrasonic image 8 in many cases when the strong ultrasonic wave generating unit 6 moves. An ultrasonic wave transmitted from the ultrasonic imaging probe 5 is mostly reflected on a surface of the costa, which makes quality of the ultrasonic image drop.

### SUMMARY OF THE INVENTION

One object of the present invention is to ameliorate the above-mentioned problems.

To that end, according to one aspect of the present invention, there is provided an ultrasonic irradiation apparatus, including an ultrasonic generating unit including a plurality of piezoelectric elements configured to irradiate an ultrasonic wave; a selection unit configured to select more than one of the piezoelectric elements among the plurality of piezoelectric elements, and configured to change the selected piezoelectric elements; and a driving unit configured to drive the selected piezoelectric elements.

According to another aspect of the present invention, there is provided an ultrasonic irradiation apparatus, including an ultrasonic generating unit including a plurality of piezoelectric elements configured to irradiate an ultrasonic wave; a first substrate including a plurality of first electrodes connected to the piezoelectric elements; and a second substrate including a plurality of second electrodes located opposite the first electrodes and a common electrode connected to plural of the second electrodes; a moving mechanism unit configured to relatively move the first substrate along a surface of the second substrate; and a driving unit configured to supply driving signals for driving the piezoelectric elements to the common electrode.

According to another aspect of the present invention, there is provided anultrasonic irradiation apparatus, including an ultrasonic generating unit including a plurality of piezoelectric elements configured to irradiate an ultrasonic wave; a plurality of switches connected to the plurality of piezoelectric elements; a controller configured to switch the plurality of switches in a predetermined pattern; and a driving unit configured to drive the piezoelectric elements in the predetermined pattern.

According to another aspect of the present invention, there is provided an ultrasonic irradiation apparatus, including an ultrasonic generating unit including a plurality of piezoelectric elements configured to irradiate an ultrasonic wave; a controller configured to set a size of an aperture defined by irradiating piezoelectric elements based on a focal distance of a ultrasonic wave irradiated from the ultrasonic generating unit; and a selection unit configured to select plural of the piezoelectric elements based on the set size of the aperture.

### BRIEF DESCRIPTION OF THE DRAWINGS

A more complete appreciation of the invention and many of the attendant advantages thereof will be readily obtained as the same becomes better understood by reference to the detailed description when considered in connection with the accompanying drawings. In the drawings:

FIGS. 1A and 1B are schematic illustrations of a conventional ultrasonic irradiation apparatus;

FIG. 2 is a block diagram of an ultrasonic irradiation apparatus according to a first embodiment of the present invention;

FIG. 3A is a top view of an ultrasonic wave generating unit;

FIG. 3B is a cross sectional view taken along line A-A of the ultrasonic wave generating unit of FIG. 3A;

FIG. 4A is a perspective view of a piezoelectric element selection circuit;

FIG. 4B is a cross sectional view of the piezoelectric element selection circuit;

FIG. 4C is a cross sectional view of the piezoelectric element selection circuit;

FIG. 5A is a top view of annular array electrodes;

FIG. 5B is a top view of piezoelectric elements;

FIG. 6A is a block diagram of a piezoelectric element driving unit;

FIG. 6B is a graph showing a relationship between delay time and a number of delay circuits;

FIG. 7 is an illustration for explaining a modification for setting focal distance;

FIG. 8 is block diagram of an ultrasonic imaging apparatus;

FIG. 9 is a flow chart for explaining a procedure for irradiating an ultrasonic wave;

FIG. 10A is an ultrasonic image when an outline of a cancer is set;

FIG. 10B is an ultrasonic image when an outline of a cancer is set;

FIG. 11A is an illustration for explaining a method for moving an irradiation range of a strong ultrasonic wave;

FIG. 11B is an illustration for explaining another method for moving an irradiation range of a strong ultrasonic wave;

FIG. 12A is an illustration for explaining a first modification for selecting a piezoelectric element irradiating a strong ultrasonic wave;

FIG. 12B is another illustration for explaining a first modification for selecting a piezoelectric element irradiating a strong ultrasonic wave;

FIG. 13A is an illustration for explaining a sound pressure distribution according to the first modification;

FIG. 13B is another illustration for explaining a sound pressure distribution according to the first modification;

FIG. 14 is a view of a piezoelectric element selection circuit according to the first modification;

FIG. 15 is a view of a piezoelectric element selection circuit according to a second modification;

FIG. 16 is a view of a piezoelectric element selection circuit according to a third modification;

FIG. 17 is a view of a piezoelectric element selection circuit according to a forth modification; and

FIG. 18 is a top view of annular array electrodes according to a modification.

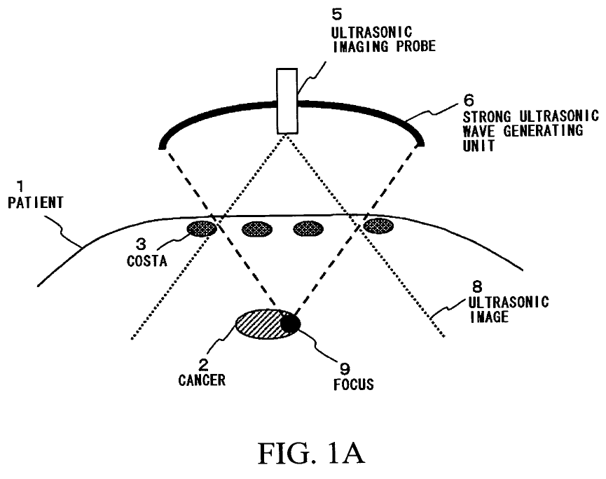

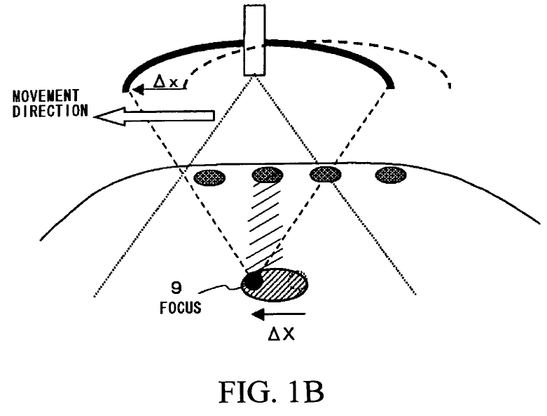

### DETAILED DESCRIPTION OF THE INVENTION

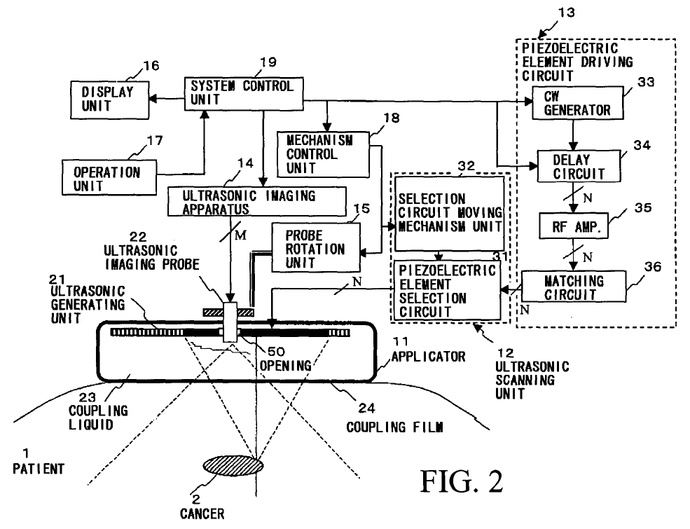

Referring now to the drawings, where like reference numbers identify the same or corresponding parts throughout the several views, embodiments of an ultrasonic irradiation apparatus according to the present invention are explained below with reference to FIG. 2 to FIG. 11. The ultrasonic irradiation apparatus of these embodiments is used for treatment by heating a cancer with a strong ultrasonic wave or used for performing an ultrasonic irradiation combination method for increasing efficiency of gene transfer. The ultrasonic irradiation apparatus has a plurality of piezoelectric elements that are two-dimensionally arranged in an applicator contacted to a patient. Some piezoelectric elements are selected from the plurality of piezoelectric elements by a piezoelectric element selection circuit separated from the applicator.

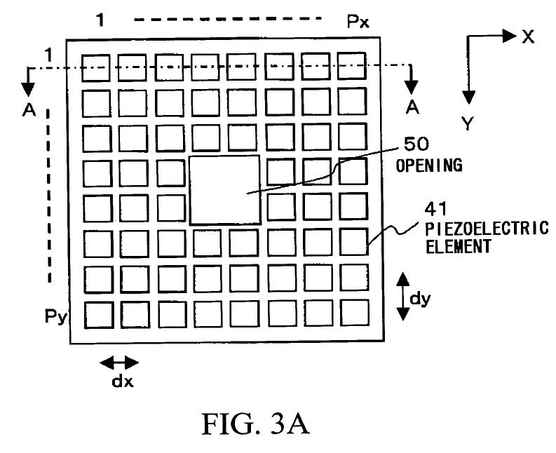

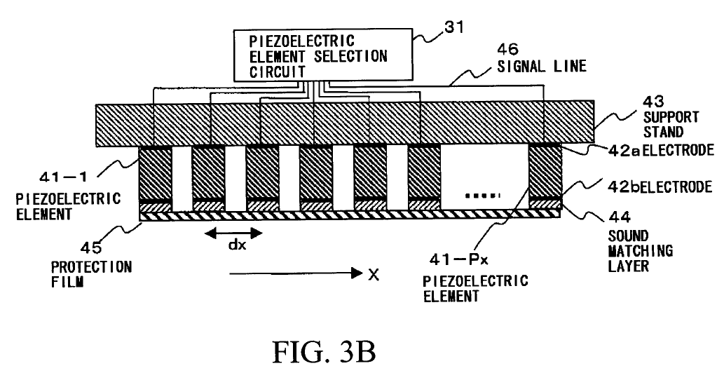

The ultrasonic irradiation apparatus is explained with reference to FIGS. 2, 3A, and 3B which show a block diagram of the ultrasonic irradiation apparatus, a top view of an ultrasonic wave generating unit, and a cross sectional view of the ultrasonic wave generating unit, respectively. A case where the ultrasonic irradiation apparatus is used for treatment by heating the cancer with a strong ultrasonic wave, is explained below. However, the ultrasonic irradiation apparatus may be similarly used for performing the ultrasonic irradiation combination method for increasing efficiency of gene transfer. The ultrasonic irradiation apparatus includes the applicator 11, which irradiates a strong ultrasonic wave to the patient 1 and monitors the effect of irradiation in an irradiation range, Further, the ultrasonic irradiation apparatus includes an ultrasonic scanning unit 12 which selects a predetermined piezoelectric elements, a piezoelectric element driving unit 13 which supplies driving signals to the selected piezoelectric elements, an ultrasonic imaging apparatus 14 which obtains an ultrasonic tomographic image including a cancer 2 irradiated by the strong ultrasonic wave from the selected piezoelectric elements, and a probe rotation unit 15 which rotates an ultrasonic imaging probe 22 rotatably attached to the applicator 11 to set a position of the ultrasonic tomographic image. Moreover, the ultrasonic irradiation apparatus has a display unit 16 which displays the ultrasonic image created by the ultrasonic imaging apparatus 14, an operation unit 17 which is used for inputting information, such as patient ID, irradiation condition, a form and a size of the cancer 2, a mechanism control unit 18 which controls the probe rotation unit 15 and a selection circuit moving mechanism unit 32 located in the ultrasonic scanning unit 12, and a system control unit 19 which controls each unit described above.

The applicator has an ultrasonic generating unit 21 which irradiates the strong ultrasonic wave, and the ultrasonic imaging probe 22 which obtains the ultrasonic image. The ultrasonic imaging probe 22 is located in an opening 50 in center of the ultrasonic generating unit 21. The ultrasonic generating unit 21 and an end of the ultrasonic imaging probe 22 are located on an upper side of the applicator 11 filled with a coupling liquid 23, such as deaeration liquid. A contact part of the applicator 11 to the patient has a coupling film 24 made from a polymer material which is flexible and has sound impedance almost equal to the patient 1 or the coupling liquid 23. That is, the strong ultrasonic wave irradiated from the ultrasonic generating unit 21 or the ultrasonic wave irradiated from the ultrasonic imaging probe 22, is efficiently transmitted to the patient 1 via the coupling film 24 and the coupling liquid 23.

The ultrasonic generating unit 21 has NX piezoelectric elements which are two-dimensionally arranged, as shown in FIG. 3A. Px piezoelectric elements are arranged in a direction X at interval dx, and Py piezoelectric elements are arranged in a direction Y at interval dy. FIG. 3B is a cross-sectional view of the ultrasonic generating unit 21 taken at section A-A in FIG. 3A. Electrode 42a and 42b which are used for supplying the driving signal are respectively located on a first face (upper face) and a second face (lower face) of the piezoelectric element 41 which includes a piezoelectric ceramic. The electrode 42a is fixed to a supporting stand 43. The electrode 42b is attached to a sound matching layer 44 for efficiently irradiating the strong ultrasonic wave. A surface of the sound matching layer 44 is covered with a protection film 45. The electrodes 42a attached to respective NX piezoelectric elements 41 are connected to a piezoelectric element selection circuit 31, mentioned below, via NX signal lines 46 used for supplying the driving signals. On the other hand, the electrodes 42b are commonly connected to a ground terminal of the ultrasonic irradiation apparatus.

The ultrasonic imaging probe 22 is used for monitoring irradiation of the strong ultrasonic wave from the ultrasonic generating unit 21 to the cancer 2 and heat efficiency of the irradiation. The ultrasonic imaging probe 22 used may be the same as the usual probe for ultrasonic diagnosis, however a sector scan probe which has small transceiver face and wide imaging range is desirable in order not to block the irradiation from the ultrasonic generating unit 21. In this embodiment, an electronic sector scan probe which electrically controls a transceiver direction of the ultrasonic wave to obtain a fan-shaped image is used as the ultrasonic imaging probe 22. M small piezoelectric elements are one-dimensionally arranged in the end of the ultrasonic imaging probe 22. Each piezoelectric element changes an electric pulse to an ultrasonic pulse to transmit the ultrasonic wave to the patient 1, and changes the ultrasonic pulse to the electric pulse to receive the ultrasonic wave from the patient 1. Since a composition of the end of the ultrasonic imaging probe 22 is similar to that of FIG. 3B, detailed explanations are omitted.

The ultrasonic scanning unit 12 has the piezoelectric element selection circuit 31 and the selection circuit moving mechanism unit 32. The piezoelectric element selection circuit 31 is a switching circuit for selecting and commonly connecting predetermined piezoelectric elements among the NX piezoelectric elements which are two-dimensionally arranged in ultrasonic generating unit 21. The piezoelectric element selection circuit 31 has a first substrate and an opposed second substrate, each of which has a plurality of electrodes. The first substrate is relatively slidable to the second substrate to supply the driving signals to arbitrary channels.

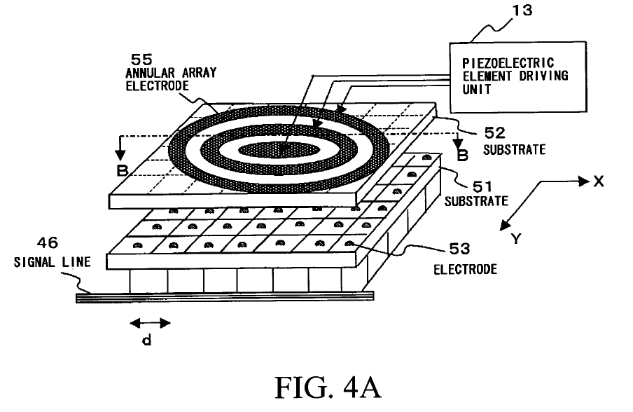

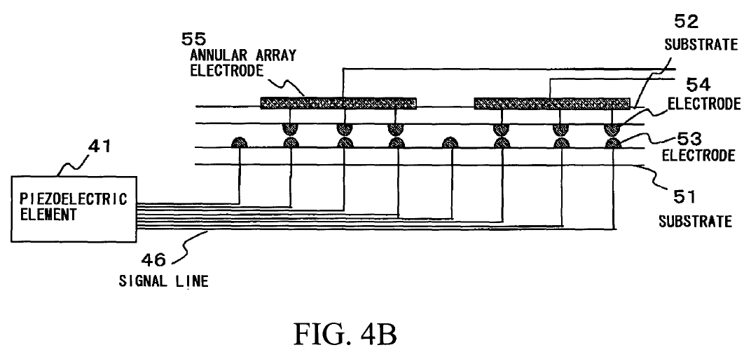

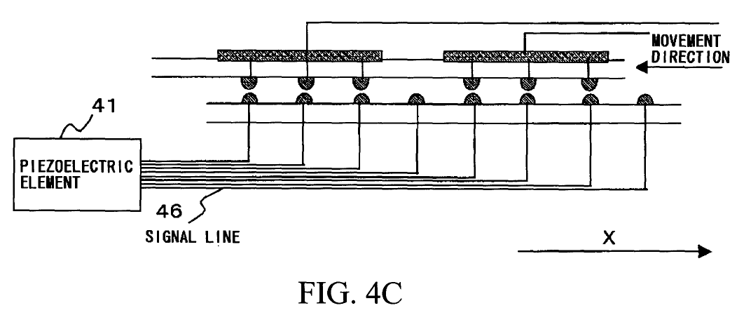

FIG. 4A shows one exemplary piezoelectric element selection circuit 31. The first substrate 51 is located opposite to the second substrate 52. Semispherical first electrodes 53 are arranged on an upper side of the first substrate 51 at interval d, and semispherical second electrodes 54 are arranged on a lower side of the second substrate 54 at interval d. The NX signal lines 46 connected to the NX piezoelectric elements 41 in the ultrasonic generating unit 21 are penetrated from a lower side of the first electrodes 51 to the first electrodes 53. That is, the piezoelectric elements are two-dimensionally arranged according to an arrangement of the first electrodes 53.

On the other hand, electrodes 55 are arranged on an upper side of the second substrate 52. The electrodes 55 are used for selecting the piezoelectric elements 41 to be driven, and are commonly connected. For example, the electrodes 55 are N-channels annular array electrodes which include a center electrode and a plurality of circular electrodes which are concentrically arranged, as shown in FIG. 4A. The N-channels electrodes are connected to N-channels outlines of the piezoelectric element driving unit 13. Although FIG. 4A shows only two circular electrodes, 5 to 15 circular electrodes are desirable.

FIG. 4B shows only a portion of the cross-section B-B. In the piezoelectric element selection circuit 31, the first substrate 51 is located near the second substrate 52. The second electrode 54 which is connected to the annular array electrode 55 via a penetration VIA through the second substrate 52, contacts the first electrode 53 of the first substrate 51, and a current can be flowed therebetween. The driving signal supplied to the annular array electrode 55 is supplied to the second electrode 54, the first electrode 53, and the piezoelectric element 41 via the signal line 46. That is, by the piezoelectric element selection circuit 31, the piezoelectric elements 41 connected to the annular array electrodes 55 are selected from the NX piezoelectric elements 41 in the ultrasonic generating unit 21. The strong ultrasonic wave formed as annular array is irradiated to the patient 1 when the ultrasonic generating unit 21 irradiates.

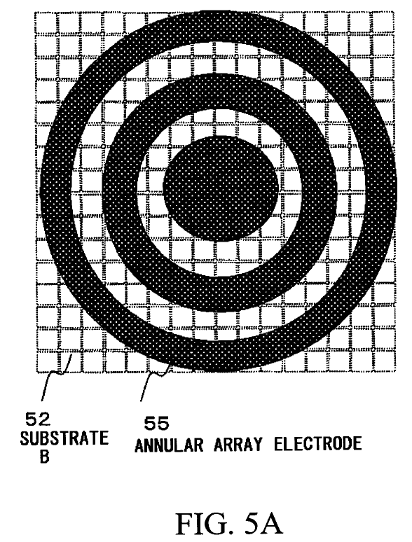

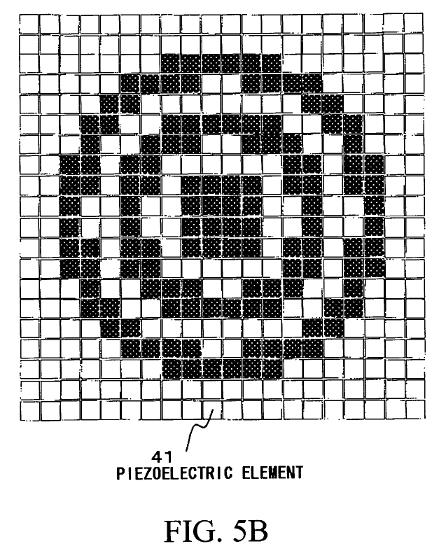

The selection circuit moving mechanism unit 32 is used for relatively moving the second substrate 52 of the piezoelectric element selection circuit 31 along a surface of the first substrate 51. When the annular array electrode 55 of the second substrate 52 mechanically moves, the position of the driven piezoelectric element 41 among the piezoelectric element 41 of the ultrasonic generating unit 21 is also moved in correspondence with the movement of the second substrate 52. Since a central position of the first electrode 53 desirably corresponds to a central position of the second electrode 54 after the movement, the relative movement distance of each direction of X or Y of the second substrate 52 may be an integral multiple of the interval d. FIG. 4C shows a sectional view at B-B section of FIG. 4A in the case the second substrate 52 moves in the X direction by the interval d relative to the first substrate 51. After the movement, next piezoelectric elements by the interval d in the X direction are selected to be driven in order to irradiate the strong ultrasonic wave. That is, by moving the second substrate 52, it is possible to select the driven piezoelectric element corresponding to the distance or the direction of the movement. In fact, a selected pattern of the annular array piezoelectric elements is mosaic-like as shown in FIG. 5B. FIG. 5A shows the annular array electrodes 55 and FIG. 5B shows the selected pattern of the piezoelectric elements 41.

The piezoelectric element driving unit 13 is used for supplying the driving signal to the piezoelectric elements so that the ultrasonic generating unit 21 irradiates the strong ultrasonic wave. The piezoelectric element driving unit 13 includes a CW generator 33 which generates a continuous wave at a frequency corresponding to the resonance frequency of the piezoelectric element 41, a delay circuit 34 which gives predetermined delay time to the continuation wave, an RF amplifier 35 which amplifies the continuous wave, and a matching circuit 36 which performs impedance matching in order to efficiently supply the output signal from the RF amplifier 35 to the piezoelectric element 41. For example, when the annular array electrodes includes the N channels electrodes, the delay circuit 34, the RF amplifier 35 and the matching circuit 36 also include N channels, and N delay times are set in the delay circuit 34. The delay circuit 34 gives the predetermined delay time to the drive signals of N channels, in order to focus the strong ultrasonic waves irradiated from the piezoelectric element 41 of the ultrasonic generating unit 21 on a desired area. The delay time is determined according to form and focal distance of the annular array electrode 55. In this embodiment, the delay time information corresponding to each focal distance is stored in the memory circuit in the system control unit 19 as a look-up table by each form of the annular array electrode 55.

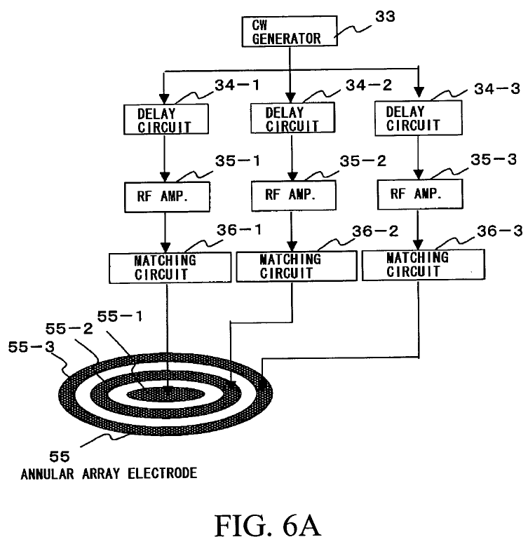

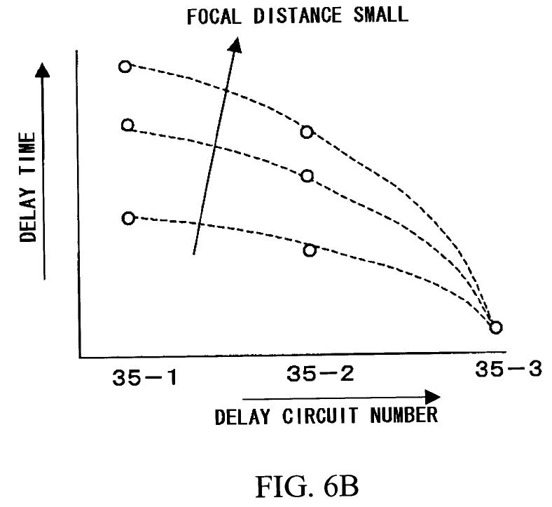

FIG. 6A shows the piezoelectric element driving unit 13 which supplies the drive signals to the annular array electrodes 55-1 to 55-3 (N=3), and FIG. 6B shows the delay time given to the drive signals of the electrode 55-1 to 55-3 by the delay circuit 34-1 to 34-3. That is, a larger delay time is set to the drive signal for the electrode 55-1 on a central part in comparison with the electrode 55-3 on an outer part. This tendency is significant when the focal distance (Fo) is small. Output signals from the delay circuits 34-1 to 34-3 of N channels are supplied to the annular array electrode 55-1 to 55-3 of the second substrate 52 in the piezoelectric element selection circuit 31 via the RF amplifier 35-1 to 35-3 and the matching circuit 36-1 to 36-3.

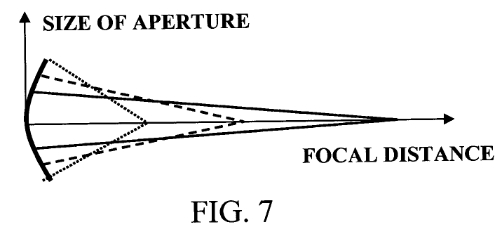

As a modification, a focal size may be changed according to the focal distance (Fo). For example, as shown in FIG. 7, when the focal distance (Fo) is large, an aperture of piezoelectric elements is small, and when the focal distance (Fo) is small, the aperture is large. In order to reduce the aperture, the electrode 55-3 shown in FIG. 6A may be OFF, and in order to enlarge the aperture, the electrode 55-3 may be ON. Otherwise, the voltage applied on the electrodes 55-1 to 55-3 may be changed according to the focal distance (Fo). For example, when the focal distance (Fo) is large, a smaller voltage may be applied on the inside electrode 55-1 than the outside electrode 55-3, and when the focal distance (Fo) is small, a larger voltage may be applied on the inside electrode 55-1 than the outside electrode 55-3. Hereby, it is possible to reduce the influence of the ultrasonic wave on the surface of the patient 1.

In another example, when the focal distance (Fo) is large, an aperture of piezoelectric elements may be large, and when the focal distance (Fo) is small, the aperture may be small. Hereby, it is possible to reduce fluctuation of the focal size to the focal distance.

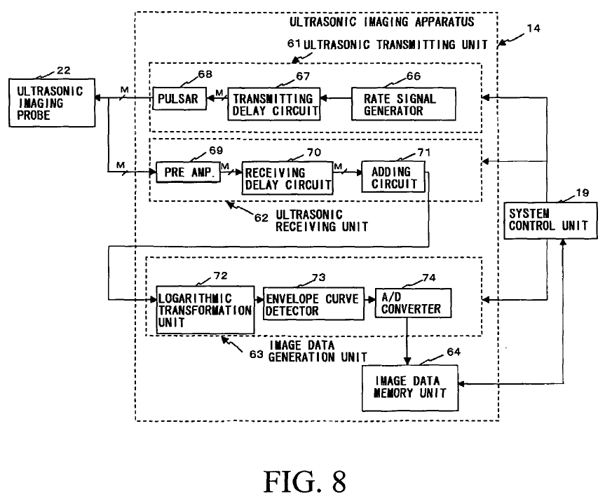

The ultrasonic imaging apparatus 14 is explained with reference to FIG. 8. The ultrasonic imaging apparatus 14 includes an ultrasonic transmitting unit 61 which generates the drive signals for irradiating the ultrasonic wave from the ultrasonic imaging probe 22 to the patient 1, and an ultrasonic receiving unit 62 which receives the ultrasonic wave from the patient 1 by the ultrasonic imaging probe 22. In addition, the ultrasonic imaging apparatus 14 includes an image data generation unit 63 which generates the ultrasonic image data based on the received signal, and an image data memory unit 64 which stores the image data. The ultrasonic transmitting unit 61 includes a rate signal generator 66, a transmitting delay circuit 67, and a pulsar 68. The rate signal generator 66 supplies a rate pulse at a rate which determines repetition cycle of the ultrasonic pulse irradiated to the patient 1 to the transmitting delay circuit 67. The transmitting delay circuit 67 includes independent delay circuits of M channels, supplies the delay time for focusing the ultrasonic wave on the predetermined depth and direction to the rate pulse from the rate signal generator, and send the delayed pulse to the pulsar 68. The pulsar 68 includes independent driving circuit of M channels. The pulsar 68 drives the piezoelectric element in the ultrasonic imaging probe 22 and creates the driving pulse for irradiating the ultrasonic wave to the patient 1.

The ultrasonic receiving unit 62 includes a preamplifier 69, a receiving delay circuit 70, and an adding circuit 71. The preamplifier 69 amplifies minute acoustic signal changed into the electric signal by the piezoelectric element, and sufficiently improves the S/N ratio. The receiving delay circuit 70 supplies the delay time for focusing the ultrasonic wave at the predetermined depth and direction to output signal from the preamplifier 69, and send the output signal to the adding circuit 71. The adding circuit 71 adds the received signals of N channels to one signal. The image data generation unit 63 includes a logarithmic transformation unit 72, an envelope curve detector 73 and an A/D converter 74. An input signal to the image data generation unit 63 is sent to the logarithmic transformation unit 72 which performs logarithmic transformation to amplitude of the input signal, in order to relatively enhance small signals. In general, the received signal from the patient 1 has amplitude with a large dynamic range of 80 dB or more. In order to display the received signal on the conventional television monitor with an about 23 dB dynamic range, the amplitude is compressed to enhance the small signal. The envelope curve detector 73 detects the envelope curve of the logarithmic transformed signal, removes ultrasonic frequency component, and detects the amplitude. The A/D converter 74 performs A/D conversion of the output signal of the envelope curve detector 73, and generates the ultrasonic image data.

The image data memory unit 64 includes a memory circuit where the ultrasonic image data generated by the image data generation unit 63 is stored. The image data obtained while changing the transceiver direction of the ultrasonic wave is stored one by one, and two-dimensional image data is created. The probe rotation unit 15 rotates the ultrasonic imaging probe 22 around a probe axis such as the area irradiated the ultrasonic wave from the ultrasonic generating unit 21 is included on the displayed ultrasonic image. The display unit 16 includes a display circuit and a CRT monitor. The display unit 16 is used for displaying the ultrasonic image produced by the ultrasonic imaging probe 22 and the ultrasonic imaging apparatus 14. The ultrasonic image data stored in the image data memory unit 64 is D/A converted and transformed to TV format data by the display circuit to be displayed on the CRT monitor. A position or beam form of the ultrasonic wave irradiated from the ultrasonic generating unit 21 may be superimposed on the ultrasonic image. A position or outline of the cancer 2 inputted by an operator with the operation unit 17, such as a mouse, may be displayed on the CRT monitor. An approximate figure of the outline of the cancer 2 may also be displayed.

The operation unit 17 includes a keyboard, a trackball, a mouse, etc. on an operation panel. The operation unit is used when the operator input patient information, cancer information, such as a position or size of the cancer and heat information, such as heating interval or heating time for each focus. The mechanism control unit 18 controls the probe rotation unit 15 and the selection circuit moving mechanism unit 32 of the ultrasonic scanning unit 12. In more detail, the mechanism control unit 18 controls movement of the selection circuit moving mechanism unit 32 according to orbit determined based on the position of the size of the cancer 2 inputted by the operation unit 17, and controls the probe rotation unit 15 such that the irradiation position of the strong ultrasonic wave is always displayed on the ultrasonic image. The system control unit 19 includes a CPU and a memory circuit, and controls each unit according to a command signal from the operation unit 17. The command and the information input with the operation unit 17 is stored in the CPU. The system control unit 19 reads the position and the size of the cancer 2 inputted with the operation unit 17, and performs elliptic approximation of the outline of the cancer 2. The system control unit 19 displays the approximate figure on the CRT monitor and sets the appropriate orbit for heating according to the cancer information. Further, the system control unit 19 calculates and displays a total time based on the heating orbit, the heating interval and the heating time for each focus.

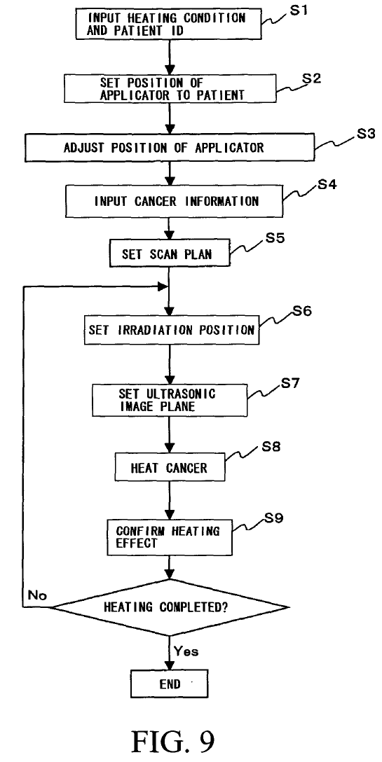

The operation for creating the ultrasonic image and for irradiating the strong ultrasonic wave is explained with reference to FIGS. 9-11. FIG. 9 shows a flow chart of the irradiation operation. The operator sets the heating condition, such as the size of the strong ultrasonic wave and the heating time for each focus, and the memory circuit 19 stores the information (Step S1). The operator sets the position of the applicator 11 such that the ultrasonic imaging probe 22 is located at appropriate position for the cancer (Step S2). At this time, the ultrasonic imaging apparatus 14 may be operated in order that the operator observe the ultrasonic image to set the position of the applicator 11.

The operation for creating the ultrasonic image is explained with reference to FIG. 8. When the ultrasonic wave is irradiated to the patient 1, the rate signal generator 66 of the ultrasonic transmitting unit 61 supplies the rate pulse which determines the repetition cycle of the ultrasonic pulse irradiated to the patient 1 to the transmitting delay circuit 67 according to the control signal from the system control unit 19. The transmitting delay circuit 67 gives the delay times for focusing the ultrasonic wave on the predetermined depth and the delay time for determining the direction ((θ1)) of the ultrasonic wave to the rate pulse, and supplies the rate pulse to the pulsar 68. The pulsar 68 drives the piezoelectric element in the ultrasonic imaging probe 22, and the ultrasonic pulse is irradiated to the patient 1.

A part of the ultrasonic wave irradiated to the patient 1 is reflected in an interface between internal organs or a tissue where sound impedance is different, the reflected part is received by the same piezoelectric element as transmitted, and the ultrasonic wave is changed into an electric signal. The received signal is amplified by the preamplifier 69, and is sent to the receiving delay circuit 70. The receiving delay circuit 70 gives the delay times for focusing and receiving the ultrasonic wave from the predetermined depth and direction ((θ1)) to the received signal, and sends the received signal to the adding circuit 71. The adding circuit 71 adds a polarity of received signals inputted from the preamplifier 69 and the receiving delay circuit 70 into one received signal, and supplies the added signal to the image data generation unit 63. To the output signal from the adding circuit 71, the logarithmic transformation, the envelope curve detection and the A/D conversion are performed, and the signal is stored in the image data memory unit 64.

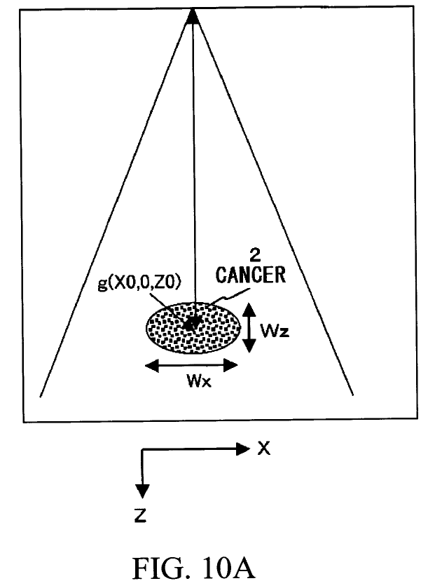

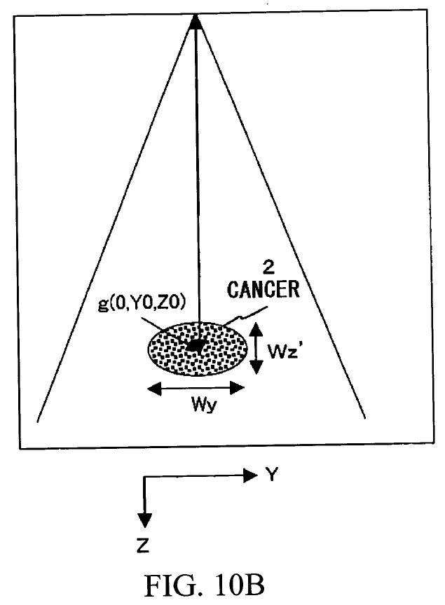

The ultrasonic wave is transmitted and received in the same procedure as the above, while the transceiver direction of the ultrasonic wave is changed by AO. That is, the system control unit 19 sequentially varies the delay time of the transmitting delay circuit 67 and the receiving delay circuit 70 according to the transceiver direction, and collects the image data. The system control unit 19 controls the image data memory unit 64 to store the image data obtained by the above-mentioned procedure, and controls the display unit 16 to display the ultrasonic image after a predetermined scan is completed. The operator adjusts the position of the applicator 11 such that the cancer 2 is located under the ultrasonic imaging probe 22, observing the ultrasonic image of the patient 1 on the CRT monitor of the display unit 16 (Step S3). FIGS. 10A-10B show the ultrasonic images displayed on the CRT monitor of the display unit 16. The piezoelectric elements of the ultrasonic imaging probe 22 are set to be one-dimensionally arranged in the X direction shown in FIG. 3A and FIG. 4A, and the ultrasonic image of X-Z plane is obtained as shown in FIG. 10A. The operator inputs the outline of the cancer 2 on the ultrasonic image by the mouse of the operation unit 19 (Step S4). The CPU of the system control unit 19 performs the elliptic approximation based on the inputted cancer outline information. Moreover, the CPU calculates a center position g (Xo, 0, Zo) of the ellipse and the maximum diameters (Wx) and (Wz) in the X and Z directions according to a top part of the ultrasonic imaging probe 22 as a basic position (X=0, Y=0, Z=0), and stores the center position and the maximum diameters in the memory circuit in the system control unit 19. When the operator inputs instruction for changing a sectional direction of the ultrasonic image with the operation unit 17, the system control unit 19 sends and instruction signal to the mechanism control unit 18. The mechanism control unit 18 supplies a rotation control signal to the probe rotation unit 15 based on the instruction signal in order to rotate the ultrasonic imaging probe 22 around a Z axis. A second ultrasonic image of Y-Z plane is displayed on the CRT monitor as shown in FIG. 10B. The operator inputs the outline of the cancer 2 by the mouse of the operation unit 17 in the similar manner as the first ultrasonic image. The CPU of the system control unit 19 performs the elliptic approximation based on the inputted cancer outline information. Moreover, the CPU calculates a center position “g” (0, Yo, Zo) of the ellipse and the maximum diameters (Wy) and (Wz) in the Y and Z directions, and stores the center position and the maximum diameters in the memory circuit in the system control unit 19. In the case that Wz is not equal to Wz′ (Wz≠Wz′), one value may be selected or an average value may be used. The system control unit 19 sets up three-dimensional moving area and orbit of the focus of the strong ultrasonic wave irradiated from the ultrasonic generating unit 21 in order to heat the cancer 2, based on the calculated center position and the size of the cancer 2 (Step S5).

Thus, a scan plan, such as the moving area and the orbit, is determined. After the determination of the scan plan, the operator inputs an irradiation start command with the operation unit 17. The system control unit 19 reads the input command and sets the delay time of the delay circuit 34 in the piezoelectric element driving unit 13 such that the focus of the strong ultrasonic wave from the ultrasonic generating unit 21 is formed in the first irradiation position g (X1, Y1, Z1) based on the scan plan. That is, the system control unit 19 reads N kinds of delay time information where the focus distance is Z1 from the look-up table in the memory circuit, and sets up the delay time of the delay circuit 34 based on the delay times information.

The system control unit 19 supplies a moving control signal to the selection circuit moving mechanism unit 32 through the mechanism control unit 18, in order to move a center position g′ (0, 0) to a position g′ (X1′, Y1′) of the annular array electrode 55. The position g′ (0, 0) of the second substrate 52 corresponds to the basis position g (0, 0, 0), namely, the top part of the ultrasonic imaging probe 22, and also corresponds to an arrangement center of the two-dimensionally arranged piezoelectric elements 41 in the ultrasonic generating unit 21. The center position g (X1′, Y′) after movement of the annular array electrode 55 corresponds to X and Y positions of the first irradiation position g (X1, Y1, Z1) (Step S6). Furthermore, the system control unit 19 supplies the rotation control signal based on the information based on the irradiation position g (X1, Y1, Z1) to the probe rotation unit 15, and rotates the ultrasonic imaging probe 22 such that the irradiation position corresponds to an ultrasonic image plane (Step S7). When the selection of the piezoelectric elements 41 at the first irradiation position, the set of the delay times of the strong ultrasonic wave irradiated to the first irradiation position and the set of the rotation angle of the ultrasonic imaging probe 22 are completed, the system control unit 19 controls the CW generator 33 of the piezoelectric element driving unit 13 to generate the continuous wave at predetermined frequency. The delay time is given to the continuous wave in the delay circuit 34 having N channels in order to focus the strong ultrasonic wave. The continuous wave passes the RF amplifier 35 and the matching circuit 36, and is supplied to the annular array electrode 55 in the second substrate 52 of the piezoelectric element selection circuit 31. The continuous wave supplied to the annular array electrode 55 is sent to the second electrode 54 located in back of the second substrate 52, and the first electrode 53 located on the first substrate 51. Further, the continuous wave passes through the signal line 46 connected to the first electrode 53, and is supplied to the piezoelectric elements 41 of the ultrasonic generating unit 21. The strong ultrasonic wave irradiated from the piezoelectric element 41 by driving the continuous wave focused on first irradiation position g (X1, Y1, Z1) set by the scan plan, and the cancer of the position is heated (Step S8).

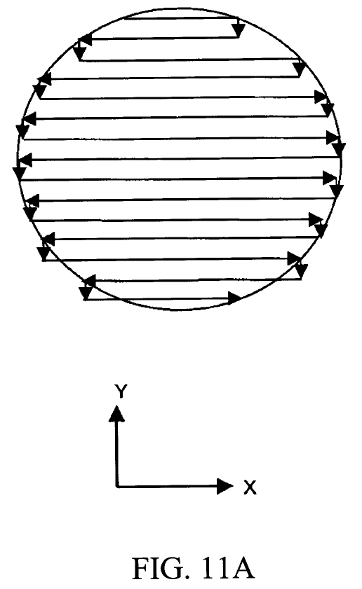

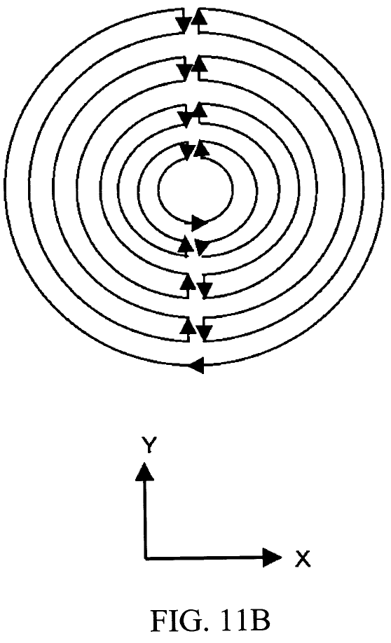

The situation of heating of the cancer by the ultrasonic generating unit 21 is obtained as ultrasonic image data by the ultrasonic imaging probe 22 and the ultrasonic imaging apparatus 14. The system control unit 19 displays the ultrasonic image on the display part 16. After the strong ultrasonic wave is irradiated to to the first irradiation position g (X1, Y1, Z1) for a predetermined time, the second irradiation position g (X2, Y2, Z2), and the third irradiation position are irradiated in order according to the scan plan. The ultrasonic imaging probe 22 is controlled to rotate, and the situation of heating the cancer is displayed on the display unit 16 in real time (Step S9). FIGS. 11A-11B show examples of movement pattern of the irradiation position of the strong ultrasonic wave, i.e., a straight line-like movement pattern and circular movement pattern, respectively. Although other movement patterns may be used, it is desirable to use a movement pattern appropriate to the ultrasonic imaging probe 22 which rotates according to the movement of the irradiation position.

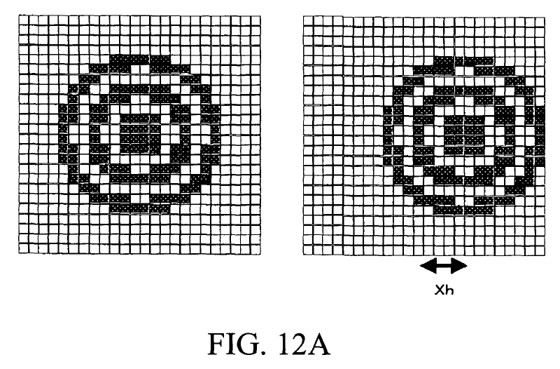

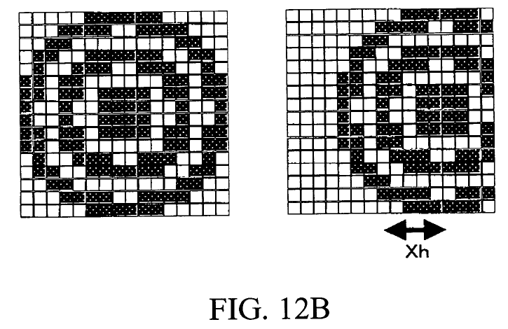

One modification of the piezoelectric element selection circuit 31 is explained with reference to FIGS. 12A-14. In this above embodiment, the piezoelectric elements 41 of the ultrasonic generating unit 21 are widely arranged. For example, as shown in FIG. 12A, when the selected piezoelectric elements 41 are shifted from a center position to an end position by Xh based on the movement of the annular array electrode 55 in the ultrasonic scanning unit 12, the selection of the piezoelectric elements shown as left side of FIG. 12A can be applied to the selection of the shifted piezoelectric elements as shown in right side of FIG. 12A. Therefore, even if the selection is made on the end position as shown in right side of FIG. 12A, a similar focus characteristic is obtained. However, in order to reduce the irradiation of the strong ultrasonic wave to a costa when a liver cancer is treated, the piezoelectric elements are selected in a narrow space as shown in FIG. 12B. In this modification, when the selected piezoelectric elements 41 are shifted to an end position by Xh, the selection is dissymmetric and misses the end position as shown right side of FIG. 12B.

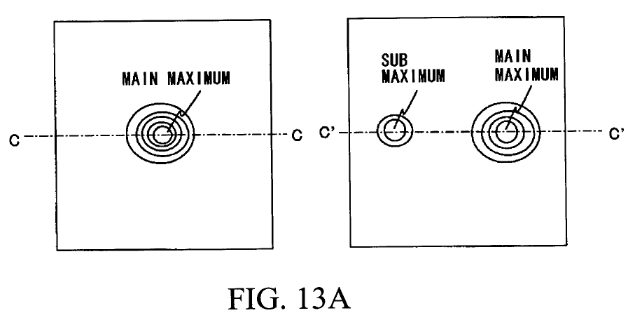

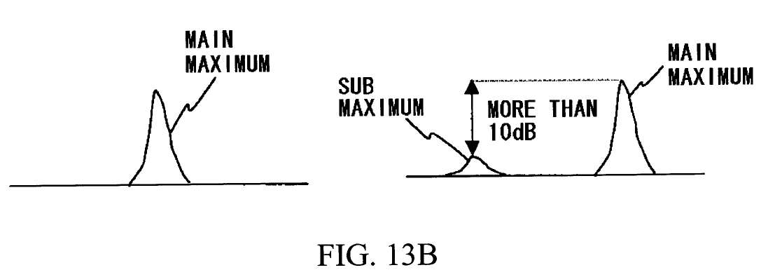

FIG. 13A shows a 2-dimensional distribution of the sound pressure of the irradiation on the central selection and the end selection. FIG. 13B shows a sound pressure value at sections C-C, and C-C′ of FIG. 13A. As shown in FIGS. 13A-13B, when the right end of the annular array pattern of the piezoelectric elements 41 is missing, a second sound pressure peak point (sub-maximum) other than the sound pressure peak point (main maximum) which is an original focus can be generated. In general, it is not so much of a problem if the difference between the sub-maximum and the main maximum is 10 dB or more. Incidentally, when the annular array whose maximum diameter is 120 mm is used for heating the cancer whose diameter is 10 mm and Xh=5 mm, the sub-maximum is permissible.

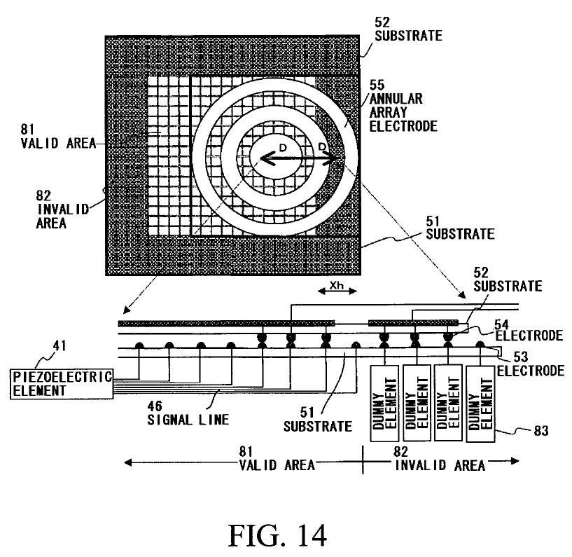

FIG. 14 shows the piezoelectric element selection circuit 31 in this modification. A relationship of positions between the first substrate 51 and the second substrate 52 is shown on an upper side of FIG. 14, and a section of D-D is shown in lower side of FIG. 14. For example, the first substrate 51 has a valid area 81 whose size is almost equal to the second substrate 52 and an invalid area around the valid area. The second substrate 52 is a square, for example, having a side length almost equal to the maximum diameter of the annular array electrode 55. The width of the invalid area is Xh which is maximum distance the annular array electrode 55 moves. The first electrode 53 on the valid area 81 is connected to the piezoelectric element 41 via the signal line 46. On the other hand, the first electrode 53 on the invalid area 82 is connected to a dummy piezoelectric element 83 which has the same impedance characteristic as the piezoelectric element 41.

For example, when the center of the annular array electrode 55 moves to right by the distance Xh, the right end part of the annular array electrode 55 is connected to the dummy piezoelectric element 83. Therefore, it is possible to reduce fluctuation of the impedance of the piezoelectric element driving unit 13, and maintain impedance matching.

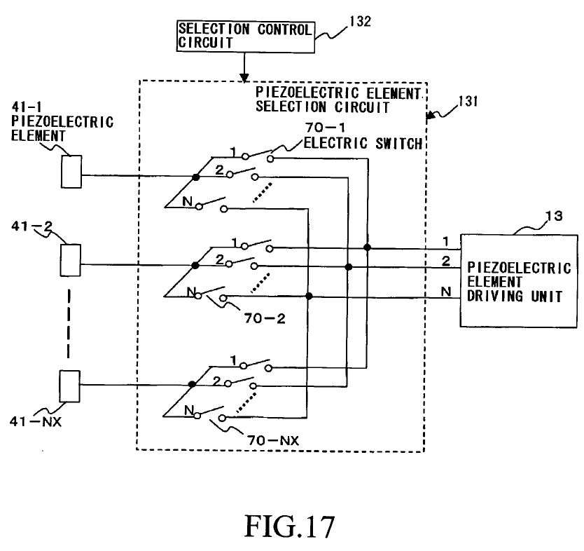

A modification of the ultrasonic scanning unit 12 of the above embodiment, is explained with reference to FIG. 17. Although the annular array electrode 55 moves to a desired direction in the above embodiment, an electronic switch is used in this modification. In FIG. 17, a piezoelectric element selection circuit 131 includes NX electronic switches 70-1 to 70-N, each of which has N channels. First terminals of the electronic switches 70-1 to 70-NX are connected to the piezoelectric elements 41-1 to 41-NX. On the other hand, second terminals of the electric switches 70-1 to 70-NX, are respectively connected to the N channels output terminals of the piezoelectric element driving unit 13. That is, piezoelectric element driving signals with N kinds of delay phases outputted from the piezoelectric element driving unit 13 are supplied to the piezoelectric elements 41 selected by the electronic switches 70-1 to 70-NX of the piezoelectric element selection circuit 131. The selected piezoelectric elements 41 irradiate the strong ultrasonic wave. A selection control circuit 132 controls the switching of the electronic switches in the piezoelectric element selection circuit 131.

As described in the above embodiment, since the ultrasonic scanning unit 12 is separated from the applicator 11 which directly contacts the patient 1, and the annular array electrode 55 of the ultrasonic scanning unit 12 moves in the desired direction, it is possible to move the irradiation position of the strong ultrasonic wave irradiated from the applicator 11 of the ultrasonic generating unit 21. Therefore, it is possible to control the irradiation position with a simple circuit. When the irradiation position is controlled, fixing the ultrasonic generating unit 21 between costae, it is possible to heat the cancer 2 located behind the costa. Moreover, when the position of the applicator 11 slightly shifts from the position of the cancer 2 at an initial stage of medical treatment, the shift may be corrected by controlling the ultrasonic scanning unit 12, and operationality improves. Furthermore, when the ultrasonic imaging probe 22 is located on a center of the applicator 11 regardless of movement of the annular array electrode 55, it is possible to reduce deterioration of the image quality due to the costa, etc.

In the noted modification, since the electric switches are used for controlling the irradiation location instead of the mechanical movement of the annular array electrode 55, the electrodes are not mechanically contacted to each other. Also by using the electric switches, since the irradiation position can be controlled while the ultrasonic generating unit 21 is located between the costae, it is possible to heat the cancer 2 located behind a costa 3 of a patient 1

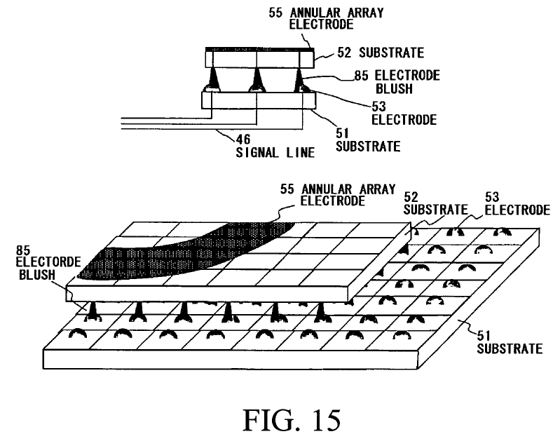

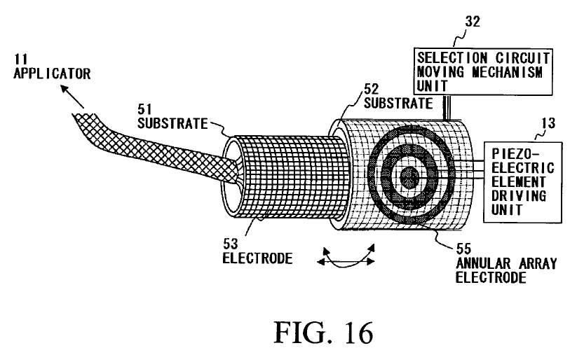

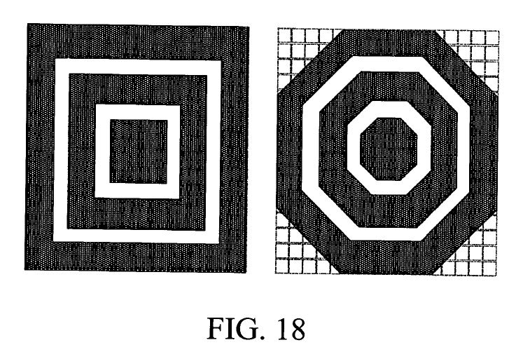

The present invention is not limited to the above embodiments, and various modifications may be made without departing from the spirit or scope of the general inventive concept. Although the first electrode 53 and the second electrode 54 shown in FIG. 4B are semispherical, other shapes may be applied. For example, as shown in FIG. 15, the first electrode 53 is semispherical and a conductive brush 85 may be used as the second electrode, or vice versa. Although the first substrate 51 and the second substrate 52 of the piezoelectric element selection circuit 31 are indicated as flat in FIG. 4, a curved surface substrate may be used. For example, the first substrate 51 and the second substrate 52 may be formed as concentric cylinders as shown in FIG. 16. In this case, the cylinder axes corresponds to each other, and the second substrate 52 covers the first substrate 51. The selection circuit moving mechanism unit 32 controls movement between the first substrate 51 and the second substrate 52 to supply the driving signal to the ultrasonic generating unit 21. Furthermore, the electrode pattern formed in the second substrate 52 may be not limited to the annular array pattern. For example, as shown in FIG. 18, a polygonal ring form may be applied or some other form may be applied. Although the piezoelectric elements 41 of the ultrasonic generating unit 21 are arranged on a one plane as shown in FIG. 3A, the piezoelectric elements may be arranged on a curved surface. Especially, when the piezoelectric element are arranged on the support stand 43 concaved to the patient 1, focal efficiency of the strong ultrasonic wave improves. Moreover, in the above embodiment, although the ellipse approximation of the outline of a cancer 2 is used, another approximation may be used, e.g., based on a selection made via the operator panel. Although the applicator is separated from the piezoelectric element selection circuit in the above embodiment, the piezoelectric element selection circuit may be located inside of the applicator. For example, electrodes may be created on the piezoelectric element as a piezoelectric element selection circuit. When the applicator and the ultrasonic imaging probe are unified, a moving area of the piezoelectric element selection circuit may be limited in order to avoid interference between the ultrasonic imaging probe and the applicator.

As described above, according to this invention, the irradiation position of the strong ultrasonic wave irradiated from the ultrasonic generating unit located near the patient is easily movable by changing selection of the piezoelectric elements. Therefore, it is possible to appropriately and easily irradiate the strong ultrasonic wave to the desired position.

Numerous modifications and variations of the present invention are possible in light of the above teachings. It is therefore to be understood that within the scope of the appended claims, the invention may be practical otherwise than as specifically described herein.

What is claimed is:

1. An ultrasonic irradiation apparatus, comprising:
an ultrasonic generating unit including a plurality of piezoelectric elements configured to irradiate an ultrasonic wave;
a selection unit configured to select more than one of the piezoelectric elements among the plurality of piezoelectric elements, and configured to change the selected piezoelectric elements; and
a driving unit configured to drive the selected piezoelectric elements.

2. The ultrasonic irradiation apparatus according to claim 1, further comprising:
an ultrasonic imaging apparatus configured to obtain ultrasonic image data at a position in an object, including a focusing device configured to focus the ultrasonic wave irradiated from the ultrasonic generating unit; and
a display unit configured to display an ultrasonic image based on the ultrasonic image data.

3. The ultrasonic irradiation apparatus according to claim 2, wherein the ultrasonic generating unit includes the plurality of piezoelectric elements two-dimensionally arranged.

4. The ultrasonic irradiation apparatus according to claim 2, wherein the selection unit comprises:
a first substrate including a plurality of first electrodes connected to the piezoelectric elements; and
a movable second substrate including a plurality of second electrodes accessible to the first electrodes.

5. The ultrasonic irradiation apparatus according to claim 4, wherein the plurality of the second electrodes are commonly connected in a predetermined pattern of arrangement.

6. The ultrasonic irradiation apparatus according to claim 5, wherein the first electrodes are connected to the piezoelectric elements via signal lines.

7. The ultrasonic irradiation apparatus according to claim 6, wherein at least one of the first electrodes and the second electrodes comprise conductive brushes.

8. The ultrasonic irradiation apparatus according to claim 5, wherein the number of the first electrodes is greater than the number of piezoelectric elements.

9. The ultrasonic irradiation apparatus according to claim 8, further comprising:
a plurality of dummy elements connected to respective of the first electrodes which are not connected to the piezoelectric elements.

10. The ultrasonic irradiation apparatus according to claim 5, wherein the first and second substrate comprise curved opposing surfaces on which said first and second electrodes are respectively arranged.

11. The ultrasonic irradiation apparatus according to claim 10, wherein:
the first substrate includes a first cylinder;
the second substrate includes a second cylinder;
the first electrodes are arranged on and outside surface of the first cylinder; and
the second electrodes are arranged on an inside surface of the second cylinder.

12. The ultrasonic irradiation apparatus according to claim 11, further comprising a moving mechanism unit configured to relatively move the first cylinder along a surface of the second cylinder.

13. The ultrasonic irradiation apparatus according to claim 2, wherein the selection unit commonly connects the piezoelectric elements in an annular array pattern.

14. The ultrasonic irradiation apparatus according to claim 2, wherein the driving unit includes a delay unit configured to set delay times to driving signals of the piezoelectric elements.

15. The ultrasonic irradiation apparatus according to claim 2, wherein the ultrasonic imaging apparatus comprises:
an ultrasonic imaging probe; and
a rotation unit configured to rotate the ultrasonic imaging probe based on the focus of the ultrasonic wave irradiated from the ultrasonic generating unit.

16. The ultrasonic irradiation apparatus according to claim 2, further comprising an operation unit configured to set a position of the focus of the ultrasonic wave irradiated from the ultrasonic generating unit.

17. The ultrasonic irradiation apparatus according to claim 16, wherein the display unit is configured to display a cancer image, and the operation unit is configured to input information on the cancer image and set the position of the focus of the ultrasonic wave based on the input information.

18. An ultrasonic irradiation apparatus, comprising:
an ultrasonic generating unit including a plurality of piezoelectric elements configured to irradiate an ultrasonic wave;
a first substrate including a plurality of first electrodes connected to the piezoelectric elements;
a second substrate including a plurality of second electrodes located opposite the first electrodes and a common electrode connected to plural of the second electrodes;
a moving mechanism unit configured to relatively move the first substrate along a surface of the second substrate; and
a driving unit configured to supply driving signals for driving the piezoelectric elements to the common electrode.

19. An ultrasonic irradiation apparatus, comprising:
an ultrasonic generating unit including a plurality of piezoelectric elements configured to irradiate an ultrasonic wave;
a plurality of switches connected to the plurality of piezoelectric elements;
a controller configured to switch the plurality of switches in a predetermined pattern; and
a driving unit configured to drive the piezoelectric elements in the predetermined pattern.

20. The ultrasonic irradiation apparatus according to claim 19, further comprising:
an ultrasonic imaging apparatus configured to obtain an ultrasonic image data of a position in an object;
said driving unit configured to focus the ultrasonic wave irradiated from the ultrasonic generating unit; and
a display unit configured to display an ultrasonic image based on the ultrasonic image data.

21. The ultrasonic irradiation apparatus according to claim 20, wherein the plurality of piezoelectric elements are two-dimensionally arranged.

22. The ultrasonic irradiation apparatus according to claim 20, wherein the controller commonly connects switches so that an annular array pattern of radiating piezoelectric elements is formed.

23. The ultrasonic irradiation apparatus according to claim 20, wherein the driving unit includes a delay unit configured to set delay times to driving signals of the piezoelectric elements.

24. The ultrasonic irradiation apparatus according to claim 20, wherein the ultrasonic imaging apparatus comprises:
an ultrasonic imaging probe; and
a rotation unit configured to rotate the ultrasonic imaging probe based on the focus of the ultrasonic wave irradiated from the ultrasonic generating unit.

25. The ultrasonic irradiation apparatus according to claim 20, further comprising an operation unit configured to set a position of the focus of the ultrasonic wave irradiated from the ultrasonic generating unit.

26. The ultrasonic irradiation apparatus according to claim 25, wherein the display unit is configured to display a cancer image, and the operation unit is configured to input information on the cancer image and set the position of the focus of the ultrasonic wave based on the input information.

27. An ultrasonic irradiation apparatus, comprising:
an ultrasonic generating unit including a plurality of piezoelectric elements configured to irradiate an ultrasonic wave;
a controller configured to set a size of an aperture defined by irradiating piezoelectric elements based on a focal distance of a ultrasonic wave irradiated from the ultrasonic generating unit; and
a selection unit configured to select plural of the piezoelectric elements based on the set size of the aperture.

28. The ultrasonic irradiation apparatus according to claim 27, wherein the controller reduces the size of the aperture when the focal distance is large, and enlarges the size of the aperture when the focal distance is small.

29. The ultrasonic irradiation apparatus according to claim 27, wherein the controller reduces the size of the aperture when the focal distance is small, and enlarges the size of the aperture when the focal distance is large.
